(19) United States
(12) Patent Application Publication
(10) Pub. No.: US 2019/0028811 A1
(43) Pub. Date: Jan. 24, 2019

(54) SCENT SPEAKER DEVICE

(71) Applicant: Jetvox Acoustic Corp., Taoyuan (TW)

(72) Inventor: To-Teng HUANG, Taoyuan (TW)

(21) Appl. No.: 15/724,675

(22) Filed: Oct. 4, 2017

(30) Foreign Application Priority Data
Jul. 21, 2017 (TW) 106210807

(51) Int. Cl. H04R 9/04; A61L 9/012

(52) U.S. Cl. CPC H04R 9/046; A61L 2209/15; A61L 9/012

(57) ABSTRACT

A scent speaker device includes a front lid, a chamber body, a voice-coil assembly, and a scent sheet. The front lid at least includes a main audio output opening defined through the front lid. The chamber body is combined with the front lid to form a protecting space. The chamber body has one or more ventilation hole for communicating the protecting space with an outside of the chamber body. The voice-coil assembly receives a driving signal to generate acoustic waves. The voice-coil assembly is disposed in the protecting space and fixed between the front lid and the chamber body. The scent sheet emits a certain scent and is fixed on an inner side of the chamber body. When the voice-coil assembly generates acoustic waves, the acoustic waves drive airflow to pass through the scent sheet to enhance the scent concentration.

### CROSS-REFERENCE TO RELATED APPLICATION

This non-provisional application claims priority under 35 U.S.C. § 119(a) to Patent Application No. 106210807 filed in Taiwan, R.O.C. on Jul. 21, 2017, the entire contents of which are hereby incorporated by reference.

### BACKGROUND

### Technical Field

The instant disclosure relates to a speaker device, in particular, to a scent speaker device.

### Related Art

3C (computer, communication, and consumer electronics) devices with multiple sensual enjoyments are gradually developed. For instance, a device may be adapted to provide audio-visual enjoyments as well as body sensations for the users. For another instance, a device may be adapted to provide audio-visual enjoyments as well as fragrant smell enjoyments for the users at the same time.

There are two types of speaker devices or earphones with fragrance in the art. One of the two types is to attach a perfume-absorbed material to a speaker device, so that the speaker device can continuously emit the fragrance. The scent concentration depends on the residual concentration of the fragrant material, and the scent concentration does not change along with the operation of the speaker device. The other approach is to assemble a small fan in the speaker device. During the operation of the speaker device, the fan blows winds outwardly to enhance the scent concentration. However, the volume of the speaker device has to be lager to assemble the fan within the speaker device. As a result, such approach is not suitable for earphones. Furthermore, the fans would lead further electricity consumption. To portable devices with limited electricity, the speaker device with the fan would have shorter operation time as compared with one without the fan. Additionally, the fan is not only a noise source but also an electromagnetic interference source, and the fan may adversely affect the audio performance of the speaker device.

### SUMMARY

The instant disclosure discloses a scent speaker device. During the operation of the scent speaker device, the scent-emitting function is achieved without additional airflow driving devices.

In one embodiment, the speaker device comprises a front lid, a chamber body, a voice coil assembly, and a scent sheet. The front lid at least has a main audio output opening defined through an inner side of the front lid and an outer side of the front lid. The chamber body is combined with the front lid to form a protecting space. The chamber body has at least one ventilation hole. The protecting space is in communication with an outside of the chamber body through the ventilation hole. The voice coil assembly receives a driving signal, and is driven by the driving signal to generate acoustic waves. The voice coil assembly is disposed in the protecting space and fixed between the front lid and the chamber body. The scent sheet is for emitting a certain scent and fixed on an inner side of the chamber body. The scent sheet is fixed on the chamber body directly or indirectly. Hence, when the voice coil assembly generates acoustic waves, the acoustic waves drive airflow to pass through the scent sheet to enhance the scent concentration.

In one embodiment, the main audio output opening is located on a middle portion of the front lid.

In one embodiment, the scent sheet covers the ventilation hole.

In one embodiment, the chamber body further comprises a detachable rear lid, the ventilation hole is on the rear lid, and the scent sheet is fixed on an inner side of the rear lid to cover the ventilation hole.

In one embodiment, the rear lid and the chamber body respectively comprises threading portions corresponding to each other, so that the rear lid is threaded with the chamber body.

In one embodiment, the speaker device further comprises a plurality of buckling members, and the rear lid is detachably fixed with the chamber body via the buckling members.

In one embodiment, the speaker device further comprises a plurality of fixing members, the fixing members pass through the rear lid from the outer side of the chamber body, and the fixing members are fixed on the chamber body, so that the rear lid is detachably fixed with the chamber body via the fixing members.

In one embodiment, the rear lid further comprises a plurality of engaging members, and the engaging members are disposed on the inner side of the rear lid to engage with the scent sheet.

In one embodiment, the speaker device comprises a replaceable cassette and the chamber body comprises a cassette-inserting hole, the scent sheet is fixed on the replaceable cassette, and the replaceable cassette is inserted into the chamber body through the cassette-inserting hole.

In one embodiment, the scent sheet is a frame body, the voice coil assembly is fixed on the scent sheet, and the scent sheet is further fixed to the chamber body.

In one embodiment, the speaker device further comprises a bracket. The voice coil assembly is fixed on the bracket, the bracket is fixed on the chamber body, and the scent sheet is fixed on the bracket.

Based on some of the foregoing embodiments, the advantages of the speaker device are described as following. The scent sheet is combined with the speaker device, so that the scent sheet becomes a portion of the speaker device. The scent sheet can be an additional component and can be replaced, or the scent sheet can be directly integrated with the front lid or the chamber body as a whole. The speaker device may be an individual earphone or a loudspeaker. During the operation of the speaker device, the acoustic waves drive airflow, so that the scent-emitting effect becomes more apparent. As a result, the speaker device can be adapted to create a certain scenario with the certain scent.

### BRIEF DESCRIPTION OF THE DRAWINGS

The disclosure will become more fully understood from the detailed description given herein below for illustration only, and thus not limitative of the disclosure, wherein:

FIG. 1 illustrates an exploded sectional view of a speaker device according to a first embodiment of the instant disclosure;

FIG. 2 illustrates a sectional view of the speaker device of the first embodiment;

FIG. 3 illustrates a lateral view of the speaker device of the first embodiment;

FIG. 4 illustrates another sectional view of the speaker device of the first embodiment;

FIG. 5 illustrates a sectional view of a speaker device according to a second embodiment of the instant disclosure;

FIG. 6 illustrates an exploded sectional view the speaker device of the second embodiment;

FIGS. 7 and 8 illustrate sectional exploded views of a speaker device according to a third embodiment of the instant disclosure;

FIG. 9 illustrates an exploded sectional view of a speaker device according to a fourth embodiment of the instant disclosure;

FIG. 10 illustrates an exploded sectional view of a speaker device according to a fifth embodiment of the instant disclosure;

FIG. 11 illustrates a sectional view of the speaker device of the fifth embodiment;

FIG. 12 illustrates an exploded sectional view of a speaker device according to a sixth embodiment of the instant disclosure;

FIG. 13 illustrates a sectional view of the speaker device of the sixth embodiment;

FIG. 14 illustrates an exploded sectional view of a speaker device according to a seventh embodiment of the instant disclosure;

FIG. 15 illustrates a sectional view of the speaker device of the seventh embodiment;

FIG. 16 illustrates an exploded sectional view of a speaker device according to an eighth embodiment of the instant disclosure; and

FIG. 17 illustrates a lateral view of the speaker device of the eighth embodiment.

### DETAILED DESCRIPTION

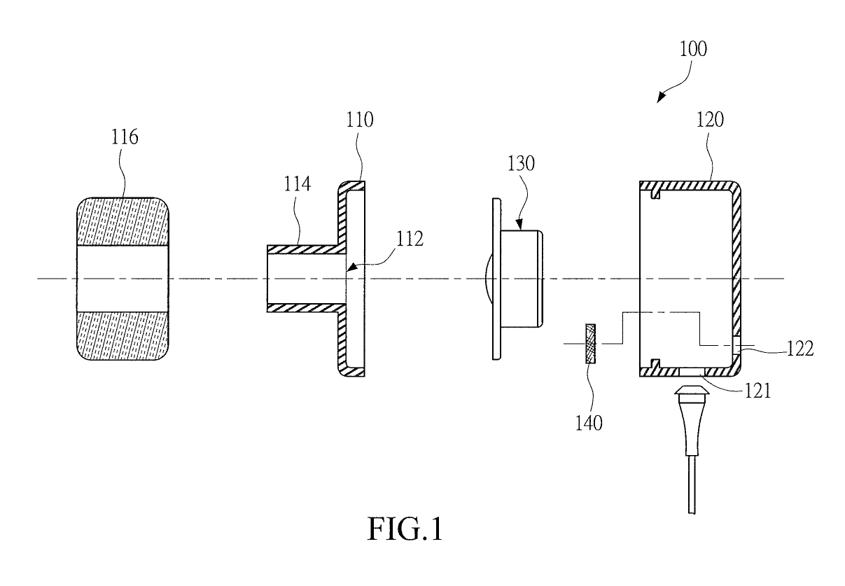

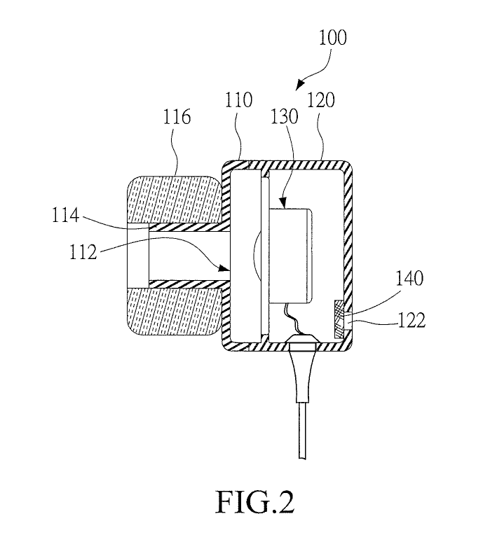

Please refer to FIGS. 1 to 3, illustrating a scent speaker device (hereinafter abbreviated as speaker) 100 according to a first embodiment of the instant disclosure. During the speaker device 100 is operated to generate acoustic waves, the emitting of a certain odor can be enhanced.

In the first embodiment, an earbud is taken as an illustrative example for the speaker device 100, but the application of the speaker device 100 is not limited thereto. The implementations of the speaker device 100 may be an earphone, a speaker, a large-size loudspeaker box, and the apparatus is not limited to a wired or a wireless apparatus.

As shown in FIGS. 1 to 3, the speaker device 100 comprises a front lid 110, a chamber body 120, a voice coil assembly 130, and a scent sheet 140.

As shown in FIGS. 1 to 3, the front lid 110 at least has a main audio output opening 112. The main audio output opening 112 is approximately located on a middle portion of the front lid 110, and the main audio output opening 112 is defined through an inner side of the front lid 110 and an outer side of the front lid 110. When the speaker device 100 is an earphone, the front lid 110 further comprises an audio output tube 114, and the audio output tube 114 extends from an outer side of the front lid 110 and communicates with the main audio output opening 112. A cushioning material member 116 further covers an outer side of the audio output tube 114. Hence, the audio output tube 114 is positioned in the external acoustic meatus of the user in a plugging manner. Accordingly, the entire speaker device 100 is positioned in the external acoustic meatus of the user.

As shown in FIGS. 1 to 3, the chamber body 120 is combined with the front lid 110, and a protecting space is formed between the chamber body 120 and the front lid 110. The chamber body 120 has at least one ventilation hole 122 defined through an inner side of the chamber body 120 and an outer side of the chamber body 120, so that the protecting space and the outside are in communication with each other through the ventilation hole 122. The ventilation hole 122 may be on any portion of the chamber body 120.

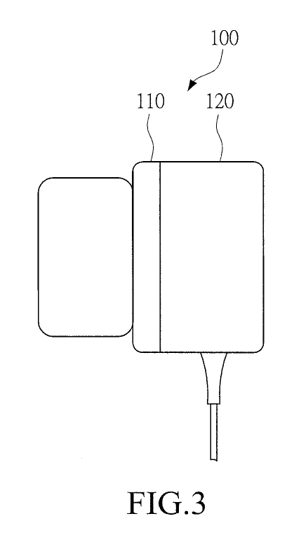

As shown in FIGS. 1 and 3, the voice coil assembly 130 is disposed in the protecting space and fixed between the front lid 110 and the chamber body 120 to form an earphone, a speaker, or a large-size loudspeaker box. The chamber body 120 may further have a wire hole 121 for allowing a signal wire to pass therethrough and entering into the protecting space, so that the signal wire can be electrically connected to the voice coil assembly 130.

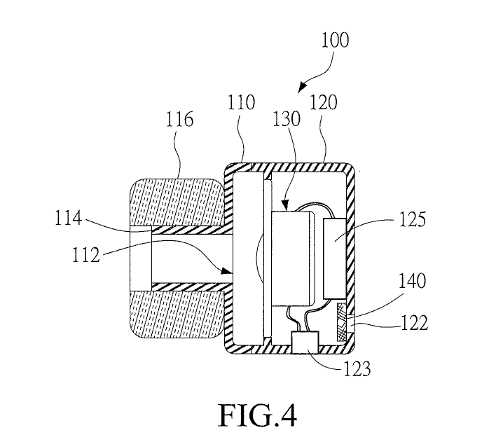

As shown in FIG. 4, when the speaker device 100 is a wireless speaker, the wire hole 121 is used for assembling an electrical connector 123. Furthermore, a battery 125 is assembled to the chamber body 120 and electrically connected to the electrical connector 123 and the voice coil assembly 130.

Please refer to FIGS. 1 to 3, the voice coil assembly 130 is used to receive a driving signal and is driven by the driving signal to generate acoustic waves. The voice coil assembly 130 may comprise, but not limited to, a vibration diaphragm, a washer, a magnet member, a yoke, a vibration coil, and a circuit board. It is understood that those components of the voice coil assembly 130 are provided as illustrative examples and are not to limit the structure or the type of the voice coil assembly 130. Loudspeaker components capable of receiving the driving signal and capable of being driven by the driving signal to generate acoustic waves can be used as the voice coil assembly 130. Hence, the components and the modifications of the voice coil assembly 130 are not described in the following paragraphs.

As shown in FIGS. 1 to 3, the scent sheet 140 is used to spread a certain scent. Materials with the certain scent are attached onto the scent sheet 140 via coating or adhering manners. For example, liquids with certain scent is coated onto the surface of the scent sheet 140 by spraying, immersion, dipping, etc., and the liquids are further absorbed into the scent sheet 140. The scent sheet 140 may be made from porous materials or fiber materials to absorb liquids with certain scent, e.g., perfumes, essential oils, or other liquids with certain scent. Hence, the scent sheet 140 has to possess great absorption abilities, and the material for making the scent sheet 140 is not limited. The material for making the scent sheet 140 may be plastics (especially porous plastics), mesh fabrics, metals (metal meshes, metal wools, and porous sintered metal sheets), fibers, or so forth. It is also understood that the source of the odor of the scent sheet 140 is not limited to the externally-added perfumes, essential oils, or liquids with certain scent. During the manufacturing of the scent sheet 140, liquids or solids with certain scent may be mixed with the materials for making the scent sheet 140. The scent sheet 140 is fixed to the voice coil assembly 130 directly or indirectly.

As shown in FIGS. 1 to 3, in the first embodiment, the scent sheet 140 is fixed to the chamber body 120; specifically, in this embodiment, the scent sheet 140 is fixed on an inner side of the chamber body 120 and the scent sheet 140 covers the ventilation hole 122. When the vibration coil of the voice coil assembly 130 receives the driving signal and is driven by the driving signal to generate acoustic waves, the acoustic waves drive airflow to pass through the scent sheet 140, to take away molecules with the certain scent, and to flow out through the ventilation hole 122. Accordingly, besides that the molecules with the certain scent on/in the scent sheet 140 can be emitted naturally, the scent-emitting effect can be enhanced when the speaker device 100 is operated to generate acoustic waves. Therefore, the speaker device 100 can be used for creating a certain scenario. Moreover, since the scent-emitting effect is not achieved by additional flow-generating device, additional energy consumption is not required when the speaker device 100 is operated, and the size or the complexity of the structure of the speaker device 100 would not be increased.

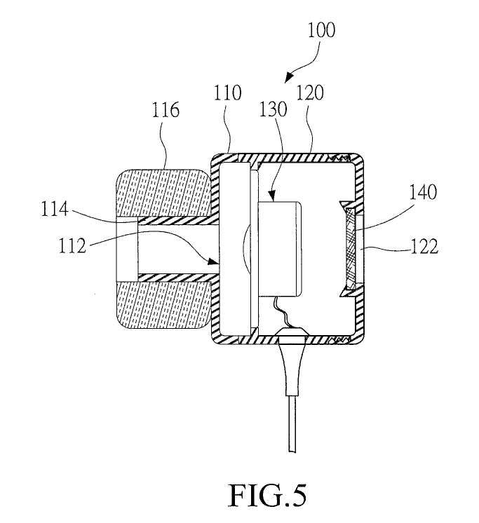

Please refer to FIG. 5, illustrating a speaker device 100 according to a second embodiment of the instant disclosure. In the second embodiment, the position and the size of the ventilation hole 122 are changed, so that the ventilation hole 122 may be further provided as a rear audio output opening of the speaker device 100.

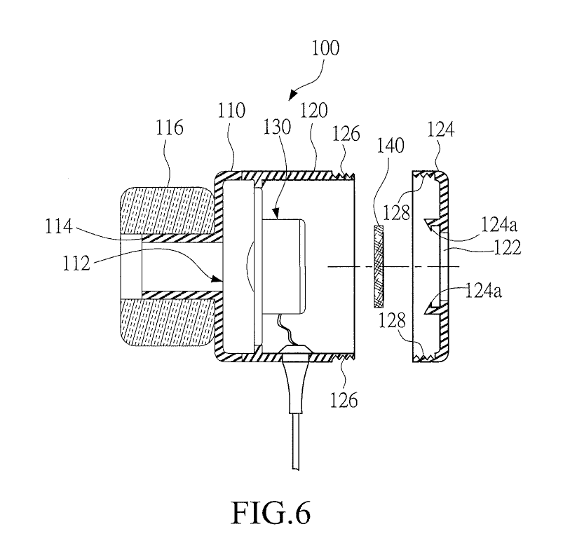

As shown in FIGS. 5 and 6, furthermore, in the second embodiment, the chamber body 120 further comprises a detachable rear lid 124. The ventilation hole 122 is on the rear lid 124, and the scent sheet 140 is fixed on an inner side of the rear lid 124 to cover the ventilation hole 122.

As shown in FIGS. 5 and 6, the rear lid 124 and the chamber body 120 respectively comprise threaded portions 126, 128 corresponding to each other, so that the rear lid 124 is threaded with the chamber body 120. Accordingly, the rear lid 124 can be detached from the chamber body 120, and the scent sheet 140 can be taken out of the rear lid 124 and exposed for applying materials with the certain odor to the scent sheet.

As shown in FIG. 6, the scent sheet 140 may be detachably fixed on the rear lid 124. For example, the rear lid 124 may further comprise a plurality of engaging members 124a, and the engaging members 124a are disposed on the inner side of the rear lid 124 to engage the scent sheet 140. Accordingly, the scent sheet 140 can be replaced easily. In this embodiment, the supplement of the certain scent is achieved by replacing the whole scent sheet 140 with a new one, rather than adding liquids with the certain odor in the original scent sheet 140.

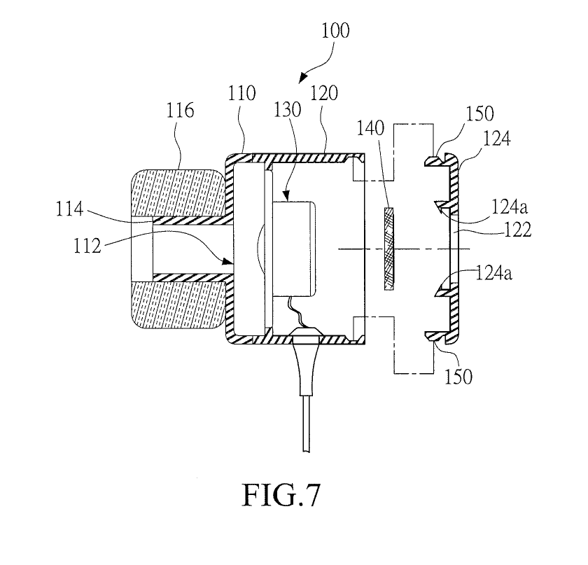

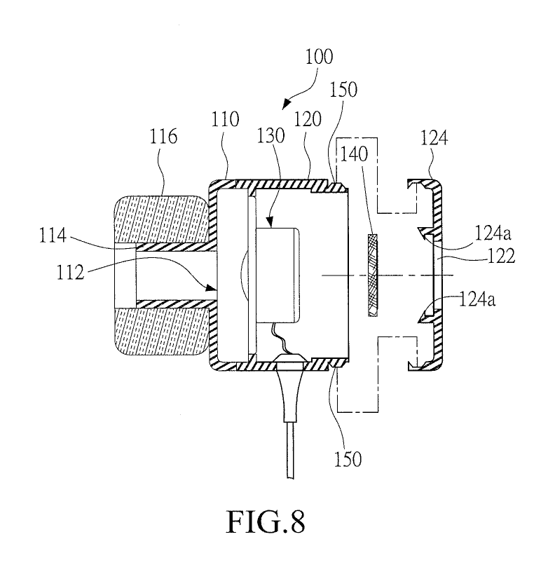

Please refer to FIGS. 7 and 8, illustrating a speaker device 100 according to a third embodiment of the instant disclosure. In the third embodiment, the threading portions 126, 128 are replaced with other features. In the third embodiment, the speaker device 100 further comprises a plurality of buckling members 150, and the rear lid 124 is detachably fixed with the chamber body 120 via the buckling members 150. The buckling members 150 extends from the rear lid 124 and the buckling members 150 are buckled with the chamber body 120, as shown in FIG. 7. Alternatively, the buckling members 150 extend from the chamber body 120 and the buckling members 150 are buckled with the rear lid 124, as shown in FIG. 8.

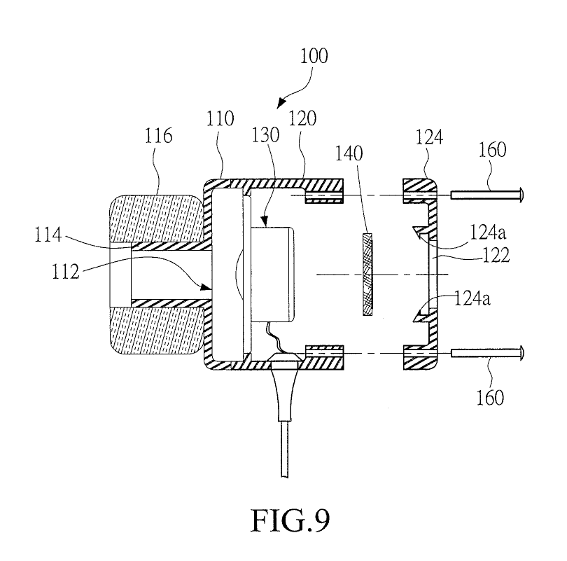

Please refer to FIG. 9, illustrating a speaker device 100 according to a fourth embodiment of the instant disclosure. In the fourth embodiment, the speaker device 100 further comprises a plurality of fixing members 160. The fixing members 160 pass through the rear lid 124 from an outer side of the chamber body 120 and the fixing members 160 are fixed on the chamber body 120, so that the rear lid 124 is detachably fixed with the chamber body 120 via the fixing members 160. In one embodiment, the fixing members 160 are bolts, and the bolts pass through the rear lid 124 and lock with the chamber body 120. The fixing members 160 may be components like, but not limited to, nails, rivets, pins, which are used to fix two objects by penetrating the two objects.

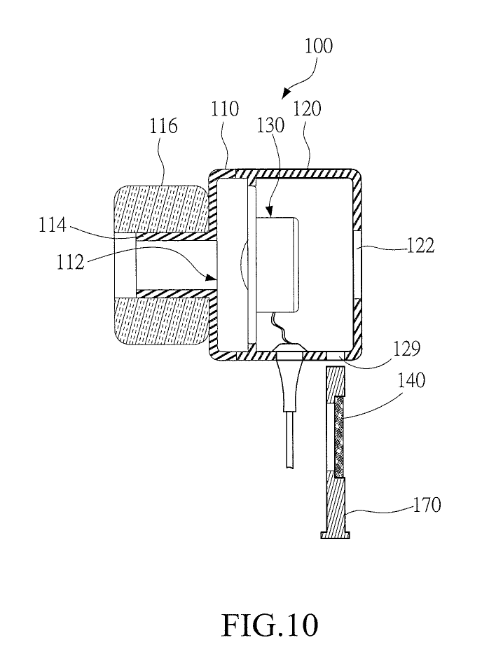

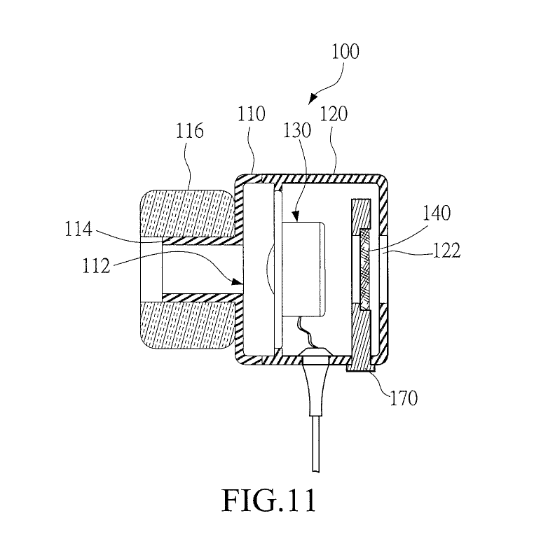

Please refer to FIGS. 10 and 11, illustrating a speaker device 100 according to a fifth embodiment of the instant disclosure. In the fifth embodiment, the speaker device 100 further comprises a replaceable cassette 170, and the chamber body 120 further comprises a cassette-inserting hole 129. The scent sheet 140 is detachably fixed on the replaceable cassette 170, the replaceable cassette 170 is inserted into the chamber body 120 through the cassette-inserting hole 129, and the scent sheet 140 is located in front of the ventilation hole 122. When the scent concentration of the scent sheet 140 is reduced, the replaceable cassette 170 can be taken out of the chamber body 120. Hence, the original scent sheet 140 can be replaced by a new one or materials with the certain scent may be applied to the scent sheet 140.

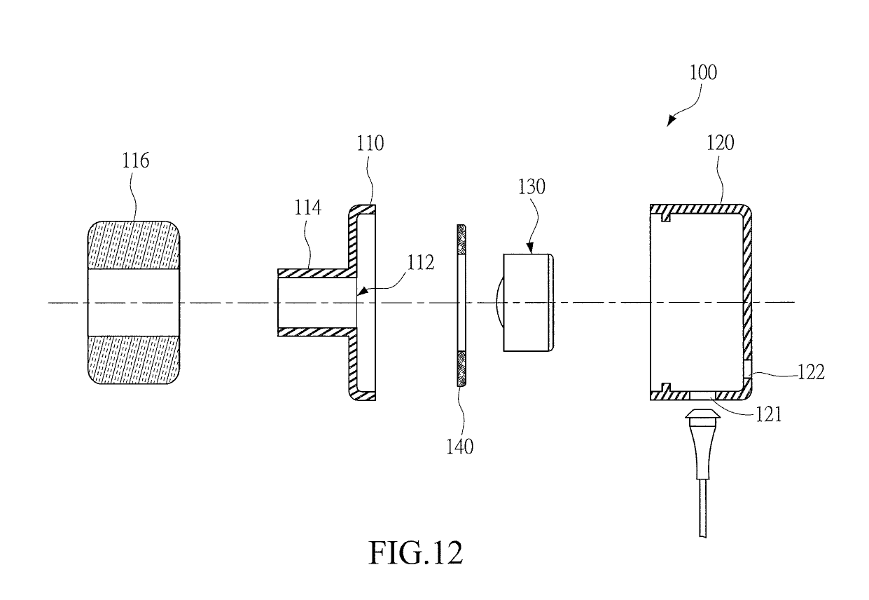

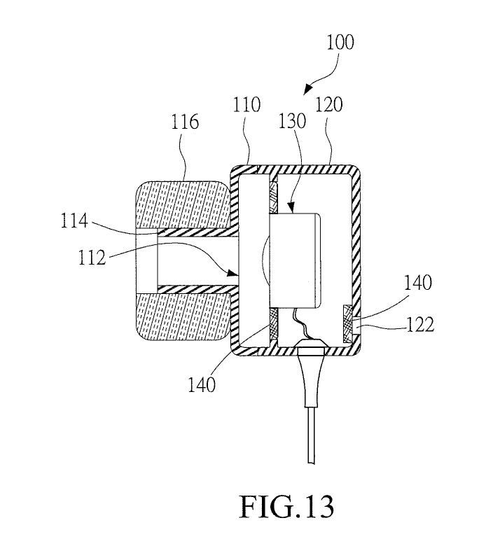

Please refer to FIGS. 12 and 13, illustrating a speaker device 100 according to a sixth embodiment of the instant disclosure. In the sixth embodiment, the scent sheet 140 is a frame body, and the voice coil assembly 130 is fixed on the scent sheet 140. The scent sheet 140 is further fixed on the chamber body 120, so that the voice coil assembly 130 is fixed between the front lid 110 and the rear lid 124.

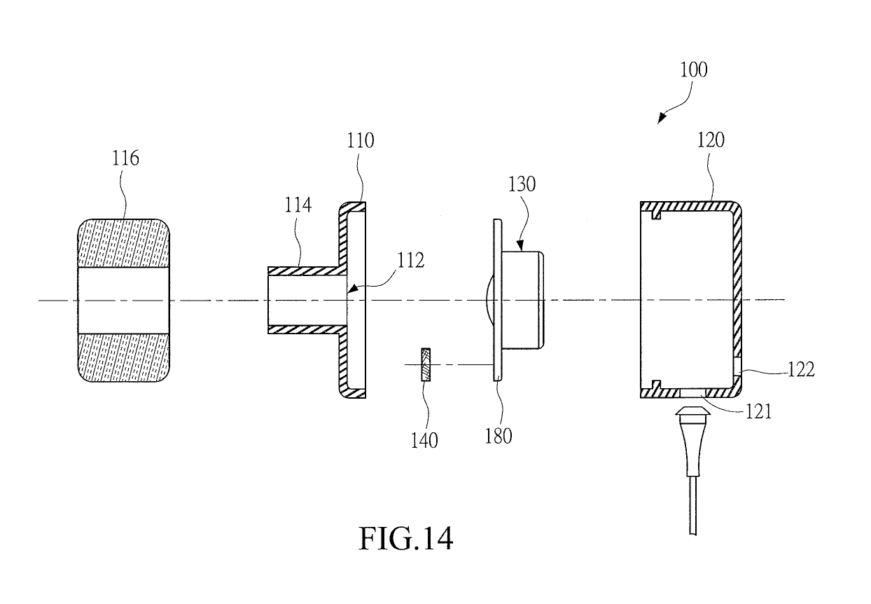

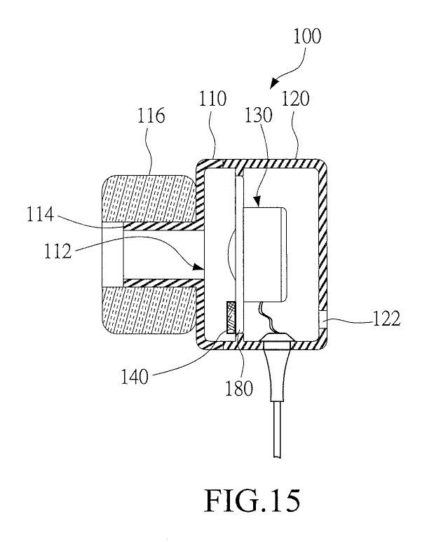

Please refer to FIGS. 14 and 15, illustrating a speaker device 100 according to a seventh embodiment of the instant disclosure. In the seventh embodiment, the speaker device 100 further comprises a bracket 180, and the voice coil assembly 130 is fixed on the bracket 180. The bracket 180 is further fixed on the chamber body 120, so that the voice coil assembly 130 is fixed between the front lid 110 and the rear lid 124. The scent sheet 140 is fixed on the bracket 180, such that the scent sheet 140 is directly fixed to the chamber body 120.

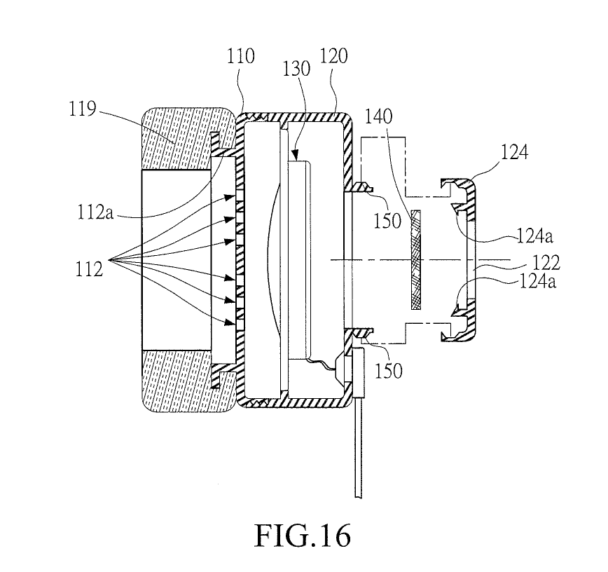

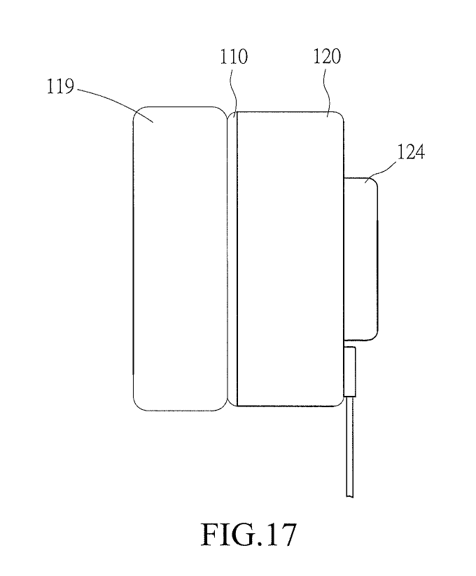

Please refer to FIGS. 16 and 17, illustrating a speaker device 100 according to an eighth embodiment of the instant disclosure. As mentioned, the speaker device 100 may be implemented in the form of an earphone, a speaker, or a loudspeaker box earphone, and the apparatus is not limited to a wired or a wireless apparatus. In the eighth embodiment, the speaker device 100 is implemented in the form of an earbud.

As shown in FIGS. 16 and 17, the speaker device 100 comprises a front lid 110, a chamber body 120, a voice coil assembly 130, and a scent sheet 140.

As shown in FIGS. 16 and 17, the front lid 110 has a plurality of main audio output openings 112 in meshed forms. Outer peripheries of the main audio output openings 112 extend to form a flange 112a. An ear cap portion encloses the flange 112a for covering user's ear, so that the main audio output openings 112 are aimed at the external acoustic meatus of the user. The chamber body 120 is combined with the front lid 110, so that a protecting space is formed between the chamber body 120 and the front lid 110. The chamber body 120 has at least one ventilation hole 122 defined through an inner side of the chamber body 120 and an outer side of the chamber body 120. As mentioned above, the chamber body 120 and the front lid 110 may be combined with each other by threading portions, buckling members, engaging members, or fixing members. In this embodiment, the chamber body 120 and the front lid 110 are combined with each other by threading portions. The voice coil assembly 130 is disposed in the protecting space and fixed between the front lid 110 and the chamber body 120.

As shown in FIGS. 16 and 17, the scent sheet 140 is fixed to the chamber body 120; specifically, the scent sheet 140 is fixed on the inner side of the chamber body 120 to cover the ventilation hole 122. The chamber body 120 further comprises a detachable rear lid 124. The ventilation hole 122 is on the rear lid 124, and the scent sheet 140 is fixed on an inner side of the rear lid 124 to cover the ventilation hole 122.

As shown in FIGS. 16 and 17, the rear lid 124 and the chamber body 120 may be combined with each other by threading portions, buckling members, engaging members, or fixing members. In this embodiment, the rear lid 124 and the chamber body 120 are combined with each other by the buckling members 150. The buckling members 150 are disposed on the chamber body 120 and the buckling members 150 are buckled with the rear lid 124. Alternatively, the buckling members 150 may extend from the rear lid 124 to be buckled with the chamber body 120. The rear lid 124 is detachable, so that the scent sheet 140 can be taken out of the rear lid 124 and exposed for applying materials with the certain odor to the scent sheet 140. It is understood that, the buckling members 150 mentioned above are provided as an illustrative purpose for the detachable assembly between components, and the embodiments are not limited thereto. Likewise, the scent sheet 140 may be detachably fixed on the rear lid 124; the detachable rear lid 124 may further comprise a plurality of engaging members 124a, and the engaging members 124a are disposed on the inner side of the rear lid 124 to engage the scent sheet 140.

It is understood that the modifications applied in the forgoing first to seventh embodiments may be also applicable to the eighth embodiment. Similarly, the speaker devices 100 of the first embodiment to the eighth embodiments may be implemented as a speaker or a loudspeaker box by modifying the structures of the front lid 110, the chamber body 120, and the rear lid 124.

Based on some of the foregoing embodiments, the scent sheet 140 is combined with the speaker device 100, so that the scent sheet 140 becomes a portion of the speaker device 100. The scent sheet 140 may be an additional component and may be replaced, or the scent sheet 140 may be directly integrated with the front lid 110 or the chamber body 120 as a whole. The speaker device 100 may be an individual earphone or a loudspeaker. During the operation of the speaker device 100, the acoustic waves drive airflow, so that the scent-emitting effect becomes more apparent. As a result, the speaker device 100 can be adapted to create a certain scenario with the certain scent.

What is claimed is:

1. A scent speaker device, comprising:
a front lid, at least having a main audio output opening, wherein the main audio output opening is defined through an inner side of the front lid and an outer side of the front lid;
a chamber body combined with the front lid to form a protecting space; wherein the chamber body has at least one ventilation hole, the protecting space is in communication with an outside of the chamber body through the ventilation hole;
a voice coil assembly receiving a driving signal and being driven by the driving signal to generate acoustic waves; wherein the voice coil assembly is disposed in the protecting space and fixed between the front lid and the chamber body; and
a scent sheet for emitting a certain scent, fixed on an inner side of the chamber body.

2. The scent speaker device according to claim 1, wherein the scent sheet covers the ventilation hole.

3. The scent speaker device according to claim 1, wherein the chamber body further comprises a detachable rear lid, the ventilation hole is on the rear lid, and the scent sheet is fixed on an inner side of the rear lid to cover the ventilation hole.

4. The scent speaker device according to claim 3, wherein the rear lid and the chamber body respectively comprises threaded portions corresponding to each other, so that the rear lid is threaded with the chamber body.

5. The scent speaker device according to claim 3, further comprising a plurality of buckling members, wherein the rear lid is detachably fixed with the chamber body via the buckling members.

6. The scent speaker device according to claim 3, further comprising a plurality of fixing members, wherein the fixing members pass through the rear lid from an outer side of the chamber body, and the fixing members are fixed on the chamber body, so that the rear lid is detachably fixed with the chamber body via the fixing members.

7. The scent speaker device according to claim 3, wherein the rear lid further comprises a plurality of engaging members, the engaging members are disposed on the inner side of the rear lid to engage with the scent sheet.

8. The scent speaker device according to claim 1, further comprising a replaceable cassette, and the chamber body further comprises a cassette-inserting hole, the scent sheet is fixed on the replaceable cassette, and the replaceable cassette is inserted into the chamber body through the cassette-inserting hole.

9. The scent speaker device according to claim 1, wherein the scent sheet is a frame body, the voice coil assembly is fixed on the scent sheet, and the scent sheet is further fixed on the chamber body.

10. The scent speaker device according to claim 1, further comprising a bracket, wherein the voice coil assembly is fixed on the bracket, the bracket is fixed on the chamber body, and the scent sheet is fixed on the bracket.
